SEQUENCE LISTING (36 sequences)

SEQ ID NO: 1 - protein, 15 aa, synthetic construct
LGIWGCSGKLICTTT

SEQ ID NO: 2 - protein, 4 aa, synthetic construct
KLIC

SEQ ID NO: 3 - protein, 7 aa, synthetic construct
GFTFSSY

SEQ ID NO: 4 - protein, 6 aa, synthetic construct
SYDGSN

SEQ ID NO: 5 - protein, 20 aa, synthetic construct
DRFSAVASTPTYHNYFYMDV

SEQ ID NO: 6 - protein, 459 aa, synthetic construct
QVQLQESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 459 aa, synthetic construct
QVQLVESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 459 aa, synthetic construct
QVQLQESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 459 aa, synthetic construct
QVQLVESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 349 aa, synthetic construct
QVQLQESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSSPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 349 aa, synthetic construct
QVQLVESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSSPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, synthetic construct
DIQMTQSPSSLSASVGDRVTITCQASQDVNNYLAWYQQKPGQAPRLLIYDASNLETGVPSRFSGSGSGTDFTLTISSLQPEDLATYYCQQYDNTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 459 aa, synthetic construct; identical to SEQ ID NO: 6

SEQ ID NO: 14 - protein, 214 aa, synthetic construct
DIQMTQSPSSLSASVGDRVTITCQASQDINNYLAWYQQKPGQAPRLLIYDASNLETGVPSRFSGSGSGTDFTLTISSLQPEDLATYYCQQYDNTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 459 aa, synthetic construct; identical to SEQ ID NO: 6

SEQ ID NO: 16 - protein, 214 aa, synthetic construct
DIQMTQSPSSLSASVGDRVTITCQASQDVNNYLAWYQQKPGQAPRLLIYDASNLETGVPSRFSGSGSGTDFTLTISSLQPEDLATYYCQQYDNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 459 aa, synthetic construct; identical to SEQ ID NO: 6

SEQ ID NO: 18 - protein, 214 aa, synthetic construct
DIQMTQSPSSLSASVGDRVTITCQASQDINNYLAWYQQKPGQAPRLLIYDASNLETGVPSRFSGSGSGTDFTLTISSLQPEDLATYYCQQYDNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 459 aa, synthetic construct; identical to SEQ ID NO: 6

SEQ ID NO: 20 - protein, 214 aa, synthetic construct
DIQMTQSPSSLSASVGDRVTITCQASQDVSNYLAWYQQKPGQAPRLLIYDASNLETGVPSRFSGSGSGTDFTLTISSLQPEDLATYYCQQYDNTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 459 aa, synthetic construct; identical to SEQ ID NO: 6

SEQ ID NO: 22 - protein, 214 aa, synthetic construct
DIQMTQSPSSLSASVGDRVTITCQASQDISNYLAWYQQKPGQAPRLLIYDASNLETGVPSRFSGSGSGTDFTLTISSLQPEDLATYYCQQYDNTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 459 aa, synthetic construct; identical to SEQ ID NO: 6

SEQ ID NO: 24 - protein, 214 aa, synthetic construct
DIQMTQSPSSLSASVGDRVTITCQASQDVSNYLAWYQQKPGQAPRLLIYDASNLETGVPSRFSGSGSGTDFTLTISSLQPEDLATYYCQQYDNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 459 aa, synthetic construct; identical to SEQ ID NO: 6

SEQ ID NO: 26 - protein, 214 aa, synthetic construct
DIQMTQSPSSLSASVGDRVTITCQASQDISNYLAWYQQKPGQAPRLLIYDASNLETGVPSRFSGSGSGTDFTLTISSLQPEDLATYYCQQYDNLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 459 aa, synthetic construct; identical to SEQ ID NO: 6

SEQ ID NO: 28 - protein, 110 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCP

SEQ ID NO: 29 - protein, 110 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 30 - protein, 110 aa, synthetic construct
KAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 110 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTSP

SEQ ID NO: 32 - protein, 110 aa, synthetic construct
PSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 33 - protein, 129 aa, synthetic construct
QVQLQESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSS

SEQ ID NO: 34 - protein, 129 aa, synthetic construct
QVQLVESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSS

SEQ ID NO: 35 - protein, 482 aa, synthetic construct
QVQLVESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 36 - protein, 469 aa, synthetic construct
QVQLVESGGGVVQPGRSLRLSCAASGFTFSSYAIHWVRQAPGKGLEWVAVISYDGSNRYYADSVKGRFTISRDNSKNTLYLQMNSLRPEDTAVYYCARDRFSAVASTPTYHNYFYMDVWGKGTTVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

(19) United States
(12) Patent Application Publication
(10) Pub. No.: US 2025/0002565 A1
(43) Pub. Date: Jan. 2, 2025

(54) HIV-BINDING PEPTIDES AND MEDICAL USE THEREOF

(71) Applicants: Joseph COTROPIA, Heath, TX (US); Gaurav CHANDRA, Heath, TX (US)

(72) Inventors: Joseph COTROPIA, Heath, TX (US); Gaurav CHANDRA, Heath, TX (US)

(21) Appl. No.: 18/692,676

(22) PCT Filed: Sep. 15, 2022
(86) PCT No.: PCT/US2022/043598

§ 371 Date: Mar. 15, 2024

Related U.S. Application Data
(60) Provisional application No. 63/245,122, filed on Sep. 16, 2021.

(51) Int. Cl. C07K 16/10; A61K 39/00; A61P 31/18

(52) U.S. Cl. CPC C07K 16/1063; A61P 31/18; A61K 2039/505; C07K 2317/569; C07K 2317/76; C07K 2317/92

(57) ABSTRACT

Described are novel HIV-binding peptides and methods of using them for treating or preventing HIV infection and/or the development of AIDS.

### CROSS-REFERENCE TO RELATED APPLICATIONS

This application claims priority to U.S. provisional application 63/245,122, filed Sep. 16, 2021, the entire contents of which are incorporated herein by reference.

### FIELD

The present disclosure relates generally to the field of human immunodeficiency virus (HIV) therapeutics, specifically peptides that bind to HIV. The disclosed HIV-binding peptides can be used in methods of treating, preventing, or reducing the risks of HIV infection, alone or in combination with other anti-HIV therapies.

### BACKGROUND

The following discussion is merely provided to aid the reader in understanding the disclosure and is not admitted to describe or constitute prior art thereto.

The causative pathogen for Acquired Immunodeficiency Syndrome (AIDS) has been shown to be the human immunodeficiency virus (HIV). The virus gains entry into certain human lymphocytes, such as T-cells and macrophages, via the CD4 receptor. Cells which have the CD4 receptor are called CD4+ cells. T-cells and macrophages are cells which play a role in cell-mediated immunity, and the surfaces of these cells have cell surface molecules, including CD4. CD4 acts as a co-receptor to a T-cell receptor (TCR) which is involved in activating the T-cells function in immunity following an antigenic introduction to the cell.

For HIV to infect human CD4+ cell, the virus must bind to first and second co-receptors to gain entry into the CD4+ cell and complete the cycle of infection. The gp120 protein of HIV is able to bind to CD4 (first co-receptor), after which the gp120 protein changes conformation. After this conformational change, CXCR4 and CCR5, other surface receptors on T-cells and macrophages (i.e., second co-receptors), can be bound by regions associated with gp120 and/or other HIV envelope ligands that are exposed upon the conformational change of gp120, such as gp41. Thus, binding of HIV to CD4 is the first step in a mechanism by which HIV infects T-cells, which in turn can lead to a compromised immune system that can manifest in sickness or death.

Neutralization of HIV prior to entry into CD4+ cells is a proposed approach to treating and preventing HIV infection. Neutralization of the virus can be achieved via at least two routes: an inhibitor binding directly to the virus that prevents the virus from binding to target CD4+ cells or an inhibitor binding to CD4+ cells, thereby preventing the virus from gaining access to the cells. Monoclonal antibodies designed to prevent the virus from binding with CD4+ cells have been developed. For example, antibodies previously patented by BioClonetics Incorporated can be used to disrupt fusion between HIV and the CD4+ cell membrane. See, e.g., U.S. Pat. Nos. 5,459,060, 5,777,074, 6,008,044, and 6,083,504. However, to date there are no clinically approved neutralizing peptides or antibody-based treatments for HIV or AIDS. Instead, current therapy regimens predominately involve administration of anti-retroviral treatments such as protease inhibitors, non-nucleoside reverse transcriptase inhibitors, integrase inhibitors, and/or nucleoside/nucleotide analogues. Unlike neutralization approaches that prevent the virus from ever entering the cells, these treatments predominately rely on a mechanism of preventing viral proliferation.

Thus, there remains a need for effective agents, compositions and methods for treating, preventing, and/or reducing the risks of HIV infection, or for targeting CD+4 cells so as to prevent HIV from binding target cells, effectively neutralizing the virus.

### SUMMARY

Described herein are peptides that bind HIV, compositions comprising the peptides, and methods using them for treating, preventing, or reducing the risks of HIV infection and/or AIDS.

In one aspect, the present disclosure provides recombinant peptides that bind to human immunodeficiency virus (HIV), comprising a heavy chain variable region comprising at least three complementarity determining regions (CDRs) including: CDR1 comprising the amino acid sequence of GFTFSSY (SEQ ID NO:3); CDR2 comprising the amino acid sequence of SYDGSN (SEQ ID NO:4); and CDR3 comprising the amino acid sequence of DRFSAVASTPTYHNYFYMDV (SEQ ID NO:5); wherein the peptide binds to an epitope of HIV comprising the amino acid sequence KLIC and prevents binding (fusion) between HIV and CD4+cells.

In some embodiments, the heavy chain variable region comprises SEQ ID NO:33 or SEQ ID NO:34.

In some embodiments, the peptide is a recombinant immunoglobulin or fragment thereof, such as an immunoglobulin heavy chain dimer or an immunoglobulin heavy chain monomer. In some embodiments, the peptide is a single-domain antibody.

In some embodiments, the peptide comprises SEQ ID NO:6 or SEQ ID NO:7, while in some embodiments, the peptide comprises SEQ ID NO:8 or SEQ ID NO:9. In some embodiments, the peptide comprises SEQ ID NO:10 or SEQ ID NO:11.

In some embodiments, the peptide comprises a CH2 domain and a CH3 domain. In some embodiments, the CH2 domain may comprise SEQ ID NO:29 or SEQ ID NO:32; and the CH3 domain may comprise SEQ ID NO:30. In some embodiments, the peptide does not comprise a CH1 domain, while in some embodiments, the peptide may comprise a CH1 domain. In those embodiments in which the peptide comprises a CH1 domain, the CH1 domain may comprise SEQ ID NO:28 or SEQ ID NO:31.

In some embodiments, the peptide comprises SEQ ID NO:33.

In some embodiments, the peptide comprises at least one constant domain from a human IgA. In some embodiments, the IgA is a secreted IgA. In some embodiments, the IgA is an IgA1 or an IgA2. In some embodiments, the peptide comprises SEQ ID NO: 35 or SEQ ID NO: 36.

In some embodiments, the epitope of HIV to which the peptide binds comprises the amino acid sequence LGIWGCSGKLICTTT (SEQ ID NO:1) or a fragment thereof comprising the amino acid sequence KLIC (SEQ ID NO:2). In some embodiments, the peptide exhibits an optical density of at least about 0.441, at least about 0.745, or at least about 1.714 in an ELISA binding assay at 1000 ng/ml of the recombinant peptide.

In some embodiments, the peptide may be conjugated to a peptide toxin, a cytotoxic drug, or a tubulin inhibitor. In some embodiments, the peptide toxin may be selected from ricin A or a fragment thereof, a Pseudomonas exotoxin or a fragment thereof; a pulchellin toxin or a fragment thereof; or gelonin. In some embodiments, the cytotoxic drug may be selected from epirubicin, gemcitabine, monomethyl auristatin E (MMAE), duocarmycin, or maytansine.

In some embodiments, the peptide is PEGylated.

In another aspect, the present disclosure provides methods of treating, preventing, or reducing the risk of HIV infection in a subject in need thereof, comprising administering to the subject a therapeutically effective amount of a recombinant peptide according to any one of the foregoing or disclosed peptides.

In some embodiments, administration of the recombinant peptide provides the subject with passive immunity to HIV. In some embodiments, the recombinant peptide is administered orally or parenterally.

In another aspect, the present disclosure provides any of the foregoing or disclosed peptides for treating, preventing, or reducing the risk of HIV infection in a subject in need thereof.

In another aspect, the present disclosure provides uses of any of the foregoing or disclosed recombinant peptides in the preparation of a medicament for treating, preventing, or reducing the risk of HIV infection in a subject in need thereof.

In another aspect, the present disclosure provides methods of preparing a recombinant peptide that binds to HIV, comprising: (a) identifying an asymptomatic patient that has been infected with HIV as a donor for obtaining immune B-lymphocytes that produce high titers of HIV-neutralizing antibodies; (b) collecting the B-lymphocytes from the patient; (c) immortalizing the B-lymphocytes; and (d) collecting antibodies produced by the heterohybridoma.

In some embodiments, immortalization of the B-lymphocytes may be performed with by fusion with a heteromyeloma cell in order to produce heterohybridoma cell. In some embodiments, the method may further comprise screening supernatants from the immortalized B-lymphocytes for HIV-binding antibodies. In some embodiments, the method may further comprise testing the antibodies for binding HIV. In some embodiments, the method may further comprise epitope mapping the antibodies that tested positive for binding to HIV. In some embodiments, purifying the antibodies from the cell culture supernatant comprises affinity chromatographic techniques. In some embodiments, the method may further comprise testing the antibodies in vitro to confirm neutralization reactivity at physiologic concentrations against HIV.

In some embodiments, a recombinant peptide that binds to human immunodeficiency virus (HIV), comprising at least three complementarity determining regions (CDRs) including: CDR1 comprising the amino acid sequence of GFTFSSY (SEQ ID NO:3); CDR2 comprising the amino acid sequence of SYDGSN (SEQ ID NO:4); and CDR3 comprising the amino acid sequence of DRFSAVASTPTYHNYFYMDV (SEQ ID NO:5); may be produced according to the foregoing method of production.

The foregoing general description and following detailed description are exemplary and explanatory and are intended to provide further explanation of the invention.

### BRIEF DESCRIPTION OF DRAWINGS

FIG. 1 shows the results of an enzyme-linked immunosorbent assay (ELISA) analyzing the binding affinity of HIV-binding peptides #9Q, #10Q, and #11Q to the 15-mer epitope (reduced with DTT for the purposes of this experiment) having the amino acid sequence of SEQ ID NO:1, which comprises a core epitope sequence of KLIC (SEQ ID NO: 2).

FIG. 2 shows the results of an ELISA binding assay analyzing the binding affinity of HIV-binding peptide #10V against the 15-mer epitope having the amino acid sequence of SEQ ID NO:1 and HIV gp41 under various conditions.

FIG. 3 shows the results of an ELISA binding assay analyzing the binding affinity of HIV-binding peptide #10Q against the 15-mer epitope having the amino acid sequence of SEQ ID NO:1 and HIV gp41 under various conditions.

FIGS. 4A-4H show the results of ELISA binding assays analyzing the binding affinity of HIV-binding peptides #1-8 against the 15-mer epitope having the amino acid sequence of SEQ ID NO:1 and HIV gp41. Specifically, FIG. 4A shows the results for HIV-binding peptide #1; FIG. 4B shows the results for HIV-binding peptide #2; FIG. 4C shows the results for HIV-binding peptide #3; FIG. 4D shows the results for HIV-binding peptide #4; FIG. 4E shows the results for HIV-binding peptide #5; FIG. 4F shows the results for HIV-binding peptide #6; FIG. 4G shows the results for HIV-binding peptide #7; and FIG. 4H shows the results for HIV-binding peptide #8.

### DETAILED DESCRIPTION

Described herein are HIV-binding peptides, compositions comprising the peptides, and methods using them, including for treating, preventing, and/or reducing the risk of HIV infection in a subject in need thereof.

### I. Definitions

As used in the description herein and the appended claims, the singular terms “a”, “an” and “the” designate both the singular and the plural, unless expressly indicated to designate the singular or unless the context clearly indicates otherwise. As used herein, the term “and/or” refers to and encompasses any and all possible combinations of one or more of the listed items, as well as each item individually (not in combination) by operation of the alternative (“or”).

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the phrases “therapeutically effective amount” and “therapeutic level” mean that dose or plasma concentration that provides the specific pharmacological effect for which the agent is administered, e.g., to reduce, ameliorate, or eliminate the symptoms or effects of HIV infection and/or reduce or eliminate viral load. It is emphasized that a therapeutically effective amount or therapeutic level may not always be effective in treating AIDS or treating or preventing HIV infection in a given subject, even though such amount or level is considered to be a therapeutically effective amount or level by those skilled in the art. For convenience only, exemplary doses, drug delivery amounts, therapeutically effective amounts, and therapeutic levels are disclosed herein. Further, the therapeutically effective amount may vary based on the route of administration and dosage form, age and weight of the subject, and/or severity of the subject's condition.

The terms “treatment” or “treating” as used herein with reference to AIDS and HIV refer to reducing, ameliorating or eliminating one or more symptoms or effects of AIDS or HIV (e.g., chronic weight loss, recurring fever/chills/night sweats, persistent diarrhea, rashes/sore/lesions, swollen lymph nodes, decreased CD4+ lymphocyte count, etc.) and/or decreasing or eliminating viral load in the subject.

The terms “individual,” “subject,” and “patient” are used interchangeably herein, and refer to any mammalian subject, including bovine, canine, feline, equine, and human subjects.

### II. HIV and AIDS

HIV is a virus spread through certain body fluids that attacks the body's immune system, specifically the CD4 cells, often called T cells. HIV reduces the number of CD4 cells (helper T4-cells) in the body. Over time, HIV can destroy so many of these cells that the body cannot fight off other infections and diseases. Opportunistic infections or cancers take advantage of the very weak immune system and signal that the person has AIDS.

When an individual is infected with HIV and does not receive treatment, the individual will typically progress through three stages of disease. Conventional HIV therapy, known as anti-retroviral therapy, can be beneficial at all stages of the disease if taken the right way, every day. Anti-retroviral treatment can slow or prevent progression from one stage to the next, and can reduce the chance of transmitting HIV to someone else. However, there is still no currently available cure or prophylactic for HIV infection.

### Stage 1: Acute HIV Infection

Within 2 to 4 weeks after infection with HIV, individuals may experience a flu-like illness, which may last for a few weeks. This is the body's natural response to infection. When individuals have acute HIV infection, they have a large amount of virus in their blood and are very contagious. But people with acute infection are often unaware that they are infected because they may not feel sick right away or at all. Treating HIV at this stage, or preventing infection altogether, would provide dramatic clinical and societal benefit.

### Stage 2: Clinical Latency (HIV Inactivity or Dormancy)

This period is sometimes called asymptomatic HIV infection or chronic HIV infection. During this phase, HIV is still active but reproduces at very low levels. People may not have any symptoms or get sick during this time. For people who are not taking medicine to treat HIV, this period can last a decade or longer, but some may progress through this phase faster. People who are taking medicine to treat HIV (e.g., anti-retroviral therapy) may be in this stage for several decades.

People can still transmit HIV to others during this phase, although people who are on anti-retroviral therapy and stay virally suppressed (having a very low level of virus in their blood) are much less likely to transmit HIV than those who are not virally suppressed. At the end of this phase, a person's viral load starts to go up, and CD4 cell count begins to go down. As this happens, the person may begin to have symptoms as the virus levels increase in the body, and the person moves into Stage 3.

### Stage 3: Acquired Immunodeficiency Syndrome (AIDS)

AIDS is the most severe phase of HIV infection. People with AIDS have such badly damaged immune systems that they may contract an increasing number of severe illnesses (i.e., opportunistic illnesses) that may be debilitating or fatal. Without treatment, people with AIDS typically survive only about 3 years. Common symptoms of AIDS include chills, fever, sweats, swollen lymph glands, weakness, and weight loss. People typically are diagnosed with AIDS when their CD4 cell count drops below 200 cells/mm³ of blood or if they develop certain opportunistic illnesses. People with AIDS can have a high viral load and may be very infectious.

### III. HIV-Binding Peptides

Disclosed herein are HIV-binding peptides that bind to a specific epitope on the HIV viral capsid protein, thereby preventing the virus from binding CD4+ cells, and compositions comprising them. In some embodiments, a pharmaceutical composition comprising such an HIV-binding peptide is administered in a therapeutically effective amount to a subject in need thereof, such as an amount effective to reduce plasma levels of HIV RNA, reduce viral load, increase CD4+ lymphocyte count, and/or reduce, ameliorate, or eliminate one or more signs or symptoms of HIV infection. The methods described herein may be used for treating, preventing, or reducing the risk of HIV infection or the development of AIDS in an individual in need thereof.

As used herein, “HIV-binding peptides” includes antibodies and antibody fragments, monomers, dimers, single-domain antibodies, and other immunoglobulin fragments, variants, or derivatives. The HIV-binding peptides disclosed herein can be obtained by any means, including from in vitro sources (e.g., a hybridoma or a cell line producing the peptide recombinantly) and in vivo sources (e.g., rodents, rabbits, humans, etc.). In some embodiments, the peptides may be produced by a heterohybridoma, as discussed in more detail below.

Without being bound by theory, it is believed that all of the HIV-binding peptides disclosed herein specifically bind to a common epitope on the HIV transmembrane protein gp41. This epitope may comprise the amino acid sequence LGIWGCSGKLICTTT (SEQ ID NO:1), or more specifically, the amino acid sequence KLIC (SEQ ID NO:2). Thus, in some embodiments, SEQ ID NO:2 may represent the minimal epitope to which the disclosed HIV-binding peptides specifically bind. Although gp41 is a dynamic structure during early replication, the epitope KLIC (SEQ ID NO: 2) is accessible on an infected cell. Accordingly, antibodies that bind this epitope may be neutralizing against HIV, and indeed, the data provided in the Example herein support this conclusion.

In general, the disclosed HIV-binding peptides comprise at least a least a portion of an immunoglobulin heavy chain. For instance, in some embodiments, the peptide may comprise a heavy chain monomer, a heavy chain dimer, or may be a single-domain antibody (i.e., a V_{H}H fragment, a “nanobody,” or a “camelid-like” antibody). A single-domain antibody may comprise or consist of a V_{H} domain, a C_{H2} domain, and a C_{H3} domain, but not a V_{K} domain or a C_{H1} domain.

Further, the disclosed HIV-binding peptides may comprise the complementarity determining regions (“CDRs”) of a heavy chain immunoglobulin. These CDRs may include a CDR1 comprising the amino acid sequence GFTFSSY (SEQ ID NO:3), a CDR2 comprising the amino acid sequence SYDGSN (SEQ ID NO:4), and a CDR3 comprising the amino acid sequence DRFSAVASTPTYHNYFYMDV (SEQ ID NO:5). In some embodiments, the disclosed HIV-binding peptides comprise a variable heavy chain amino acid sequence of QVQLQESGGGVVQPGRSLRLSCAASGFTFSSYAIHWVRQAPGKGLEWVAVISYDGSNR YYADSVKGRFTISRDNSKNTLYLQMNSLRPEDTAVYYCARDRFSAVASTPTYHNYFYM DVWGKGTTVTVSS (SEQ ID NO:33). Heavy chain sequences of exemplary HIV-binding peptides are disclosed in Table 1 below.

TABLE 1
Heavy Chain Sequences of HIV-Binding Peptides

| Peptide | | SEQ |
|---|---|---|
| ID | Heavy Chain Amino Acid Sequence | ID NO: |
| #9Q | QVQLQESGGGVVQPGRSLRLSCAASGFTFSSYAIHWVRQAPGKGLE | 6 |
| | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEA | |
| | LHNHYTQKSLSLSPGK | |
| #9V | QVQLVESGGGVVQPGRSLRLSCAASGFTFSSYAIHWVRQAPGKGLE | 7 |
| | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEA | |
| | LHNHYTQKSLSLSPGK | |
| #11Q | QVQLQESGGGVVQPGRSLRLSCAASGFTFSSYAIHWVRQAPGKGLE | 8 |
| | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSSDKTHTSPPSP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVESCSVMHEA | |
| | LHNHYTQKSLSLSPGK | |
| #11V | QVQLVESGGGVVQPGRSLRLSCAASGFTFSSYAIHWVRQAPGKGLE | 9 |
| | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSSDKTHTSPPSP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEA | |
| | LHNHYTQKSLSLSPGK | |
| #10Q | QVQLQESGGGVVQPGRSLRLSCAASGFTFSSYAIHWVRQAPGKGLE | 10 |
| | WVAVISYDGSNRYYADSVKGRFTISRDNSKNTLYLQMNSLRPEDTAV | |
| | YYCARDRFSAVASTPTYHNYFYMDVWGKGTTVTVSSPCPAPELLGG | |
| | PSVFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHYT | |
| | QKSLSLSPGK | |
| #10V | QVQLVESGGGVVQPGRSLRLSCAASGFTFSSYAIHWVRQAPGKGLE | 11 |
| | WVAVISYDGSNRYYADSVKGRFTISRDNSKNTLYLQMNSLRPEDTAV | |
| | YYCARDRFSAVASTPTYHNYFYMDVWGKGTTVTVSSPCPAPELLGG | |
| | PSVFLFPPKPKDTLMISRTPEVTCVVVDVSHEDPEVKFNWYVDGVEVHNA | |
| | KTKPREEQYNSTYRVVSVLTVLHQDWLNGKEYKCKVSNKALPAPIEKTISK | |
| | AKGQPREPQVYTLPPSREEMTKNQVSLTCLVKGFYPSDIAVEWESNGQPE | |
| | NNYKTTPPVLDSDGSFFLYSKLTVDKSRWQQGNVFSCSVMHEALHNHYT | |
| | QKSLSLSPGK | |

Heavy and light chain complementarity determining regions (CDRs) are shown in bold, underlined text. CDR annotation was made according to IMGT numbering. Constant regions are denoted in italicized, underlined text.

In accordance with some embodiments, the disclosed HIV-binding peptides do not require an immunoglobulin light chain in order to bind HIV. In accordance with some embodiments, the disclosed HIV-binding peptides comprise both a heavy and light chain. In accordance with some embodiments, the disclosed HIV-binding peptides are full antibodies (e.g., complete IgGs). Exemplary heavy and light chain sequences that may be present in exemplary HIV-binding antibodies are disclosed in Table 2 below.

TABLE 2
Heavy and Light Chain Sequences of HIV-Binding Peptides

| Peptide | | SEQ |
|---|---|---|
| ID | Heavy or Light Chain Amino Acid Sequence | ID NO: |
| #1 Light | DIQMTQSPSSLSASVGDRVTITCQASQDVNNYLAWYQQKPGQAPRLL | 12 |
| Chain | IYDASNLETGVPSRFSGSGSGTDFTLTISSLQPEDLATYYCQQYDNTP | |
| | YTFGQGTKLEIKRTVAAPSVFIFPPSDEQLKSGTASVVCLLNNFYPREAKV | |
| | QWKVDNALQSGNSQESVTEQDSKDSTYSLSSTLTLSKADYEKHKVYACEVT | |
| | HQGLSSPVTKFNRGEC | |
| #1 Heavy | QVQLQESGGGVVQPGRSLRLSCAASGFTFSSYAIHWVRQAPGKGLE | 13 |
| Chain | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEA | |
| | LHNHYTQKSLSLSPGK | |
| #2 Light | DIQMTQSPSSLSASVGDRVTITCQASQDINNYLAWYQQKPGQAPRLLI | 14 |
| Chain | YDASNLETGVPSRFSGSGSGTDFTLTISSLQPEDLATYYCQQYDNTPY | |
| | TFGQGTKLEIKRTVAAPSVFIFPPSDEQLKSGTASVVCLLNNFYPREAKVQ | |
| | WKVDNALQSGNSQESVTEQDSKDSTYSLSSTLTLSKADYEKHKVYACEVTH | |
| | QGLSSPVTKSFNRGEC | |
| #2 Heavy | QVQLQESGGGVVQPGRSLRLSCAASGFTFSSYAIHWVRQAPGKGLE | 15 |
| Chain | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEA | |
| | LHNHYTQKSLSLSPGK | |
| #3 Light | DIQMTQSPSSLSASVGDRVTITCQASQDVNNYLAWYQQKPGQAPRLL | 16 |
| Chain | IYDASNLETGVPSRFSGSGSGTDFTLTISSLQPEDLATYYCQQYDNLP | |
| | YTFGQGTKLEIKRTVAAPSVFIFPPSDEQLKSGTASVVCLLNNFYPREAKV | |
| | QWKVDNALQSGNSQESVTEQDSKDSTYSLSSTLTLSKADYEKHKVYACEVT | |
| | HQGLSSPVTKSFNRGEC | |
| #3 Heavy | QVQLQESGGGVVQPGRSLRLSCAASGFTFSSYAIHWVRQAPGKGLE | 17 |
| Chain | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEA | |
| | LHNHYTQKSLSLSPGK | |
| #4 Light | DIQMTQSPSSLSASVGDRVTITCQASQDINNYLAWYQQKPGQAPRLLI | 18 |
| Chain | YDASNLETGVPSRFSGSGSGTDFTLTISSLQPEDLATYYCQQYDNLPY | |
| | TFGQGTKLEIKRTVAAPSVFIFPPSDEQLKSGTASVVCLLNNFYPREAKVQ | |
| | WKVDNALQSGNSQESVTEQDSKDSTYSLSSTLTLSKADYEKHKVYACEVTH | |
| | QGLSSPVTKSFNRGEC | |
| #4 Heavy | QVQLQESGGGVVQPGRSLRLSCAASGFTFSSYAIHWVRQAPGKGLE | 19 |
| Chain | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEA | |
| | LHNHYTQKSLSLSPGK | |
| #5 Light | DIQMTQSPSSLSASVGDRVTITCQASQDVSNYLAWYQQKPGQAPRLL | 20 |
| Chain | IYDASNLETGVPSRFSGSGSGTDFTLTISSLQPEDLATYYCQQYDNTP | |
| | YTFGQGTKLEIKRTVAAPSVFIFPPSDEQLKSGTASVVCLLNNFYPREAKV | |
| | QWKVDNALQSGNSQESVTEQDSKDSTYSLSSTLTLSKADYEKHKVYACEVT | |
| | HQGLSSPVTKSFNRGEC | |
| #5 Heavy | QVQLQESGGGVVQPGRSLRLSCAASGFTFSSYAIHWVRQAPGKGLE | 21 |
| Chain | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVESCSVMHEA | |
| | LHNHYTQKSLSLSPGK | |
| #6 Light | DIQMTQSPSSLSASVGDRVTITCQASQDISNYLAWYQQKPGQAPRLLI | 22 |
| Chain | YDASNLETGVPSRFSGSGSGTDFTLTISSLQPEDLATYYCQQYDNTPY | |
| | TFGQGTKLEIKRTVAAPSVFIFPPSDEQLKSGTASVVCLLNNFYPREAKVQ | |
| | WKVDNALQSGNSQESVTEQDSKDSTYSLSSTLTLSKADYEKHKVYACEVTH | |
| | QGLSSPVTKSFNRGEC | |
| #6 Heavy | QVQLQESGGGVVQPGRSLRLSCAASGFTFSSYAIHWVRQAPGKGLE | 23 |
| Chain | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEA | |
| | LHNHYTQKSLSLSPGK | |
| #7 Light | DIQMTQSPSSLSASVGDRVTITCQASQDVSNYLAWYQQKPGQAPRLL | 24 |
| Chain | IYDASNLETGVPSRFSGSGSGTDFTLTISSLQPEDLATYYCQQYDNLP | |
| | YTFGQGTKLEIKRTVAAPSVFIFPPSDEQLKSGTASVVCLLNNFYPREAKV | |
| | QWKVDNALQSGNSQESVTEQDSKDSTYSLSSTLTLSKADYEKHKVYACEVT | |
| | HQGLSSPVTKSFNRGEC | |
| #7 Heavy | QVQLQESGGGVVQPGRSLRLSCAASGFTFSSYAIHWVRQAPGKGLE | 25 |
| Chain | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVFSCSVMHEA | |
| | LHNHYTQKSLSLSPGK | |
| #8 Light | DIQMTQSPSSLSASVGDRVTITCQASQDISNYLAWYQQKPGQAPRLLI | 26 |
| Chain | YDASNLETGVPSRFSGSGSGTDFTLTISSLQPEDLATYYCQQYDNLPY | |
| | TFGQGTKLEIKRTVAAPSVFIFPPSDEQLKSGTASVVCLLNNFYPREAKVQ | |
| | WKVDNALQSGNSQESVTEQDSKDSTYSLSSTLTLSKADYEKHKVYACEVTH | |
| | QGLSSPVTKSFNRGEC | |
| #8 Heavy | QVQLQESGGGVVQPGRSLRLSCAASGFTFSSYAIHWVRQAPGKGLE | 27 |
| Chain | WVAVISYDGSNRYYADSVKGRFTISRDNSKNTLYLQMNSLRPEDTAV | |
| | YYCARDRFSAVASTPTYHNYFYMDVWGKGTTVTVSSASTKGPSVFP | |
| | LAPSSKSTSGGTAALGCLVKDYFPEPVTVSWNSGALTSGVHTFPAVLQSSG | |
| | LYSLSSVVTVPSSSLGTQTYICNVNHKPSNTKVDKRVEPKSCDKTHTCPPCP | |
| | APELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPA | |
| | PIEKTISKAKGQPREPQVYTLPPSREEMTKNQVSLTCLVKGFYPSDIAVEW | |
| | ESNGQPENNYKTTPPVLDSDGSFFLYSKLTVDKSRWQQGNVESCSVMHEA | |
| | LHNHYTQKSLSLSPGK | |

Heavy and light chain complementarity determining regions (CDRs) are shown in bold, underlined text. CDR annotation was made according to IMGT numbering. Constant regions are denoted in italicized, underlined text.

Human, partially humanized, fully humanized, and chimeric versions of the HIV-binding peptides disclosed herein can be made by methods known in the art, such as using a transgenic animal (e.g., a mouse) wherein one or more endogenous immunoglobulin gene sequences are replaced with one or more human immunoglobulin gene sequences. Examples of transgenic mice wherein endogenous antibody genes are effectively replaced with human antibody genes include, but are not limited to, the HUMAB-MOUSE™, the Kirin TC MOUSE™, and the KM-MOUSE™ (see, e.g., Lonberg, Nat. Biotechnol., 23(9): 1117-25 (2005), and Lonberg, Handb. Exp. Pharmacol., 181: 69-97 (2008)).

Monoclonal antibodies (mAbs) and fragments thereof based on the HIV-binding peptides disclosed herein may be obtained by methods known in the art, for example, by fusing antibody-producing cells with immortalized cells to obtain a hybridoma, and/or by generating mAbs from mRNA extracted from bone marrow, B cells, and/or spleen cells of immunized animals using combinatorial antibody library technology and/or by isolating monoclonal antibodies from serum from subjects immunized with a peptide antigen from HIV, such as a peptide antigen comprising SEQ ID NO:1 or SEQ ID NO:2.

Recombinant versions of the HIV-binding peptides disclosed herein may be obtained by methods known in the art, for example, using phage display technologies, yeast surface display technologies (Chao et al., Nat. Protoc., 1(2): 755-68 (2006)), mammalian cell surface display technologies (Beerli et al., PNAS, 105(38): 14336-41 (2008), and/or by expressing or co-expressing component polypeptides, such as heavy and light chain polypeptides. Other techniques for making peptides and antibodies are known in the art, and can be used to obtain versions of the HIV-binding peptides disclosed herein.

Typically, an antibody consists of four polypeptides: two identical copies of a heavy (H) chain polypeptide and two copies of a light (L) chain polypeptide. Typically, each heavy chain contains one N-terminal variable (V_{H}) region and three C-terminal constant (C_{H}1, C_{H}2 and C_{H}3) regions, and each light chain contains one N-terminal variable (V_{L}) region and one C-terminal constant (C_{L}) region. The variable regions of each pair of light and heavy chains form the antigen binding site of an antibody, however, some of the disclosed peptides may comprise a heavy chain without a light chain.

The term “antibody fragment,” as used herein, refers to one or more portions of a HIV-binding antibody that exhibits the ability to bind HIV, particularly on or within the epitope of SEQ ID NO:1 or SEQ ID NO:2. Examples of binding fragments include (i) Fab fragments (monovalent fragments consisting of the V_{L}, V_{H}, C_{L} and C_{H1} domains); (ii) F(ab′)₂ fragments (bivalent fragment comprising two Fab fragments linked by a disulfide bridge at the hinge region); (iii) Fd fragments (comprising the V_{H} and C_{H1} domains); (iv) Fv fragments (comprising the V_{L} and V_{H} domains of a single arm of an antibody), (v) dAb fragments (comprising a V_{H} domain); and (vi) isolated complementarity determining regions (CDR), e.g., V_{H} CDR3. Other examples include single chain Fv (scFv) constructs. See e.g., Bird et al., Science, 242:423-26 (1988); Huston et al., Proc. Natl. Acad. Sci. USA, 85:5879-83 (1988). Other examples include HIV-binding domain immunoglobulin fusion proteins comprising (i) a HIV-binding domain polypeptide (such as a heavy chain variable region, a light chain variable region, or a heavy chain variable region fused to a light chain variable region via a linker peptide) fused to an immunoglobulin hinge region polypeptide, (ii) an immunoglobulin heavy chain C_{H2} constant region fused to the hinge region, and (iii) an immunoglobulin heavy chain C_{H3} constant region fused to the C_{H2} constant region, where the hinge region may be modified by replacing one or more cysteine residues with, for example, serine residues, to prevent dimerization.

As noted above, some of the disclosed HIV-binding peptides may comprise a light chain, while others may not. Similarly, some of the disclosed HIV-binding peptides may comprise a CH1 region, while others may not. For instance, in some embodiments, a disclosed HIV-binding peptide may comprise or consist of a V_{H} domain, a C_{H2} domain, and a C_{H3} domain. In some embodiments, a disclosed HIV-binding peptide may comprise or consist of a V_{H} domain, a C_{H1} domain, a C_{H2} domain, and a C_{H3} domain. In some embodiments, the constant domains may comprise one or more modifications, such as an amino acid substitution. Exemplary constant domains are shown in Table 3 below.

TABLE 3
Exemplary Constant Domains of HIV-Binding Peptides

| Peptide | | SEQ |
|---|---|---|
| ID | Constant Domain Amino Acid Sequence | ID NO: |
| C_{H1} | ASTKGPSVFPLAPSSKSTSGGTAALGCLVKDYFPEPVTVSWNSGALTS | 28 |
| | GVHTFPAVLQSSGLYSLSSVVTVPSSSLGTQTYICNVNHKPSNTKVDK | |
| | RVEPKSCDKTHTCP | |
| C_{H2} | PCPAPELLGGPSVFLFPPKPKDTLMISRTPEVTCVVVDVSHEDPEVKFN | 29 |
| | WYVDGVEVHNAKTKPREEQYNSTYRVVSVLTVLHQDWLNGKEYKC | |
| | KVSNKALPAPIEKTIS | |
| C_{H3} | KAKGQPREPQVYTLPPSREEMTKNQVSLTCLVKGFYPSDIAVEWESN | 30 |
| | GQPENNYKTTPPVLDSDGSFFLYSKLTVDKSRWQQGNVFSCSVMHEA | |
| | LHNHYTQKSLSLSPGK | |
| C_{H1} | ASTKGPSVFPLAPSSKSTSGGTAALGCLVKDYFPEPVTVSWNSGALTS | 31 |
| Variant | GVHTFPAVLQSSGLYSLSSVVTVPSSSLGTQTYICNVNHKPSNTKVDK | |
| | RVEPKSSDKTHTSP | |
| C_{H2} | PSPAPELLGGPSVFLFPPKPKDTLMISRTPEVTCVVVDVSHEDPEVKFN | 32 |
| Variant | WYVDGVEVHNAKTKPREEQYNSTYRVVSVLTVLHQDWLNGKEYKC | |
| | KVSNKALPAPIEKTIS | |

In some embodiments, an HIV-binding peptide as disclosed herein is derived from a human IgG1 antibody, a human IgG2 antibody, a human IgG3 antibody, or a human IgG4 antibody. In some embodiments, an HIV-binding peptide as disclosed herein may be derived from a class of antibody selected from IgG, IgM, IgA, IgE, and IgD. That is, the disclosed HIV-binding peptides may comprise all or part of the constant regions, framework regions, or a combination thereof of an IgG, IgM, IgA, IgE, or IgD antibody. For instance, in some embodiments, an HIV-binding peptide comprising an IgG1 immunoglobulin structure may be modified to replace (“switch”) the IgG1 structure with the corresponding structure of another IgG-class immunoglobulin or an IgM, IgA, IgE, or IgD immunoglobulin. This type of modification or switching may be performed in order to augment the neutralization functions of the peptide, such as antibody dependent cell cytotoxicity (ADCC) and complement fixation (CDC). A person of ordinary skill in the art will understand that, for example, a recombinant IgG1 immunoglobulin structure can be “switched” to the corresponding regions of immunoglobulin structures from other immunoglobulin classes, such as recombinant secretory IgA1 or recombinant secretory IgA2, such as may be useful for topical application onto the skin or mucosal surfaces, e.g., vaginal and rectal surfaces, which would allow the peptide to be more easily incorporated into vaginal gels or condoms. For example, immunoglobulin IgA structures are known to have applications in protective immune surveillance directed against invasion of infectious diseases at mucosal membrane surfaces, which makes such structures suitable for methods of using the disclosed HIV-binding peptides in such contexts, e.g., for preventing HIV infection or the spread of HIV from one individual to another.

A “switched” antibody or “class switched” antibody includes an antibody that comprises a HCDR1 comprising the amino acid sequence GFTFSSY (SEQ ID NO:3), a HCDR2 comprising the amino acid sequence SYDGSN (SEQ ID NO:4), and a HCDR3 comprising the amino acid sequence DRFSAVASTPTYHNYFYMDV (SEQ ID NO:5) and comprises one or more constant regions from, for example IgA1 or IgA2. Additionally or alternatively, a “switched” antibody or “class switched” antibody includes an antibody that comprises any one of the disclosed heavy chain variable regions (e.g., amino acids 1-120 of any one of SEQ ID NOs:6-11), light chain variable regions, or both and comprises one or more constant regions from, for example IgA1 or IgA2. In some embodiments, the IgA1 or IgA2 is a secretory IgA. In some embodiments the IgA is monomeric, while in some embodiments, the IgA is dimeric.

For example, a class switched IgA1 may comprise an amino acid sequence of QVQLVESGGGVVQPGRSLRLSCAASGFTFSSYAIHWVRQAPGKGLEWVAVISYDGSNR YYADSVKGRFTISRDNSKNTLYLQMNSLRPEDTAVYYCARDRFSAVASTPTYHNYFYM DVWGKGTTVTVSSASPTSPKVFPLSLCSTQPDGNVVIACLVQGFFPQEPLSVTWSESGQG VTARNFPPSQDASGDLYTTSSQLTLPATQCLAGKSVTCHVKHYTNPSQDVTVPCPVPST PPTPSPSTPPTPSPSCCHPRLSLHRPALEDLLLGSEANLTCTLTGLRDASGVTFTWTPSSGK SAVQGPPERDLCGCYSVSSVLPGCAEPWNHGKTFTCTAAYPESKTPLTATLSKSGNTFR PEVHLLPPPSEELALNELVTLTCLARGFSPKDVLVRWLQGSQELPREKYLTWASRQEPS QGTTTFAVTSILRVAAEDWKKGDTFSCMVGHEALPLAFTQKTIDRLAGKPTHVNVSVV MAEVDGTCY (SEQ ID NO: 35). A class switched antibody may be a heavy chain monomer (i.e., one chain comprising SEQ ID NO: 35) or a heavy chain dimer (i.e., two chains comprising SEQ ID NO: 35).

Similarly, a class switched IgA2 may comprise an amino acid sequence of QVQLVESGGGVVQPGRSLRLSCAASGFTFSSYAIHWVRQAPGKGLEWVAVISYDGSNR YYADSVKGRFTISRDNSKNTLYLQMNSLRPEDTAVYYCARDRFSAVASTPTYHNYFYM DVWGKGTTVTVSSASPTSPKVFPLSLDSTPQDGNVVVACLVQGFFPQEPLSVTWSESGQ NVTARNFPPSQDASGDLYTTSSQLTLPATQCPDGKSVTCHVKHYTNPSQDVTVPCPVPP PPPCCHPRLSLHRPALEDLLLGSEANLTCTLTGLRDASGATFTWTPSSGKSAVQGPPERD LCGCYSVSSVLPGCAQPWNHGETFTCTAAHPELKTPLTANITKSGNTFRPEVHLLPPPSE ELALNELVTLTCLARGFSPKDVLVRWLQGSQELPREKYLTWASRQEPSQGTTTFAVTSIL RVAAEDWKKGDTFSCMVGHEALPLAFTQKTIDRMAGKPTHVNVSVVMAEVDGTCY (SEQ ID NO: 36). A class switched antibody may be a heavy chain monomer (i.e., one chain comprising SEQ ID NO: 36) or a heavy chain dimer (i.e., two chains comprising SEQ ID NO:36).

Such class switched antibodies may provide a protective immunological defense against initial exposure to or infection by HIV virus at mucosal surfaces, such as occurs in the passage of HIV from mother to child during breast feeding. HIV mucosal infection plays a central role not only in virus transmission but also in AIDS pathogenesis, and may effect mucosal surfaces of the gastrointestinal tract early on by depleting it of CD4+ T helper cells independently of the virus transmission route. Although current antiretroviral therapy helps controlling HIV infection in some patients, it cannot eradicate the virus from the human host. Therefore, the disclosed switched antibodies, which may provide topical pre-exposure prophylaxis, represent a promising approach to prevent HIV transmission.

In some embodiments, the HIV-binding peptide is a mammalian, human, humanized, or chimeric peptide. In some embodiments, the disclosed HIV-binding peptides comprise one or more mutations that make the peptide more suitable in a therapeutic context.

In some embodiments, an HIV-binding peptide may comprise one or more mutations, alterations, or modifications, such as one or more mutations, alterations, or modifications that improve one or more properties or functions of the peptide. Such mutations, alterations, or modifications may comprise, for example, changes to the Fc region to increase the ability of the peptide to mediate cellular cytotoxicity functions like antibody dependent cell cytotoxicity (ADCC), antibody dependent cell mediated phagocytosis (ADCP), and/or complement fixation (CDC). A wide number of mutations to the Fc domain that enhance binding to Fc receptors have been reported, for example, S239D/A330L/I332E, F243L, and G236A. Additionally or alternatively, mutations to the Fc region that increase the circulating half-life of a disclosed HIV-binding peptide may be incorporated into the structure. For example, mutations to engineer the pH-dependent interaction of the Fc domain with FcRn to increase affinity at pH 6.0 while retaining minimal binding at pH 7.4, can increase half-life and improve efficacy under physiological conditions. Exemplary mutations that may be incorporated in order to enhance C1q receptor or Fc receptor binding are shown in the table below.

TABLE 4
Potential Fc Mutations

| Mutation | Mechanism of Action | Effect |
|---|---|---|
| S267E | Enhance C1q binding | Increase CDC |
| H268F/S324T | Enhance C1q and Fc | Increase CDC, ADCC, ADCP |
| | receptor binding | |
| H268F/S324T/ | Enhance C1q and Fc | Increase CDC, ADCC, ADCP |
| G236A/I332E | receptor binding | |
| H268F/S324T/ | Enhance C1q and Fc | Increase CDC, ADCC, ADCP |
| G236D/I332E | receptor binding | |
| K326A/E333A | Enhance C1q and Fc | Increase CDC, ADCC |
| | receptor binding | |
| E345R | Enhance multimerization | Increase CDC, ADCC, ADCP |
| M252Y/S254T/ | Enhanced Fc receptor | Increased half-life |
| T256E | binding at pH 6.0 | |
| T250Q/M428L | Enhanced Fc receptor | Increased half-life |
| | binding at pH 6.0 | |
| P230S/N315D/ | Enhanced Fc receptor | Increased half-life |
| M428L/N434Y | binding at pH 6.0 | |

In some embodiments, the disclosed HIV-binding peptide may be conjugated to polyethylene glycol (PEG), which may increase the half-life and decrease the potential immunogenicity of the peptide.

In some embodiments, a HIV-binding peptide as disclosed herein comprises one or more substitutions, insertions, or deletions. For example, in some embodiments, an HIV-binding peptide comprises a heavy chain with at least about 85%, about 86%, about 87%, about 88%, about 89%, about 90%, about 91%, about 92%, about 93%, about 94%, about 95%, about 96%, about 97%, about 98%, about 99%, or about 100% identity to one or more of the heavy chain sequences disclosed in Tables 1 and 2. In some embodiments, an HIV-binding peptide comprises heavy and light chains with at least 85%, 86%, 87%, 88%, 89%, 90%, 91%, 92%, 93%, 94%, 95%, 96%, 97%, 98%, 99%, or 100% identity to any of the corresponding heavy and light chain sequence pairs disclosed in Table 2.

In some embodiments, the HIV-binding peptides disclosed herein bind to HIV gp41 (e.g., SEQ ID NO:1 of gp41) with a high affinity. As shown in the Examples section below, the novel HIV-binding peptides of Tables 1 and 2 can efficiently bind to HIV with an affinity that will prevent binding and entry of HIV into CD4+ cells. As also shown in the Examples section below, disclosed HIV-binding peptides have been shown to bind both reduced (linear) and oxidized (cyclic) versions of SEQ ID NO:1. The affinity values reported below were determined by ELISA using BIACORE® technology. Other methodology for determining binding affinity also can be used, such as equilibrium dialysis, or surface plasmon resonance biosensor.

Binding affinity can be expressed in terms of optical density (OD) derived from testing in an indirect ELISA assay, such as a BIACORE® assay. In some embodiments, the HIV-binding peptides described herein have an OD in an indirect ELISA of from about 0.250 to about 2.0 at 1000 ng/ml of competing protein. In some embodiments, the HIV-binding peptides have an OD in an indirect ELISA of at least about 0.250, 0.275, 0.300, 0.325, 0.350, 0.375, 0.400, 0.425, 0.450, 0.475, 0.500, 0.525, 0.550, 0.575, 0.600, 0.625, 0.650, 0.675, 0.700, 0.725, 0.750, 0.775, 0.800, 0.825, 0.850, 0.875, 0.900, 0.925, 0.950, 0.975, 1.000, 1.250, 1.275, 1.300, 1.325, 1.350, 1.375, 1.400, 1.425, 1.450, 1.475, 1.500, 1.525, 1.550, 1.575, 1.600, 1.625, 1.650, 1.675, 1.700, 1.725, 1.750, 1.775, 1.800, 1.825, 1.850, 1.875, 1.900, 1.925, 1.950, 1.975, or 2.000, at 1000 ng/ml of competing protein. In some embodiments, the HIV-binding peptides have an OD in an indirect ELISA of at least about 0.441, at least about 0.745, or at least about 1.714 at 1000 ng/ml of competing protein.

In some embodiments, a substitution at the 5 position of the heavy chain variable sequence (i.e., SEQ ID NO:33) from Q (glutamine) to V (valine), thus resulting in the sequence QVQLVESGGGVVQPGRSLRLSCAASGFTFSSYAIHWVRQAPGKGLEWVAVISYDGSNR YYADSVKGRFTISRDNSKNTLYLQMNSLRPEDTAVYYCARDRFSAVASTPTYHNYFYM DVWGKGTTVTVSS (SEQ ID NO:34). This Q5V substitution may improve binding. For instance, as shown in Example 3, below, HIV-binding peptide #10V bound more tightly to both the reduced and oxidized peptide epitope of SEQ ID NO:1 compares to #10Q.

In some embodiments, the HIV-binding peptides disclosed herein have a K_{D} for HIV gp41 of less than 100 nM. For example, in some embodiments, the HIV-binding peptides have a K_{D} for HIV gp41 of less than about 1.5×10⁻⁷, less than about 1.0×10⁻⁷, less than about 0.5×10⁻⁷, less than about 9.5×10⁻⁸, less than about 9.0×10⁻⁸, less than about 8.5×10⁻⁸, less than about 8.0×10⁻⁸, less than about 7.5×10⁻⁸, less than about 7.0×10⁻⁸, less than about 6.5×10⁻⁸, less than about 6.0×10⁻⁸, less than about 5.5×10⁻⁸, less than about 5.0×10⁻⁸, less than about 4.5×10⁻⁸, less than about 4.0×10⁻⁸, less than about 3.5×10⁻⁸, less than about 3.0×10⁻⁸, less than about 2.5×10⁻⁸, less than about 2.0×10⁻⁸, less than about 1.5×10⁻⁸, less than about 1.0×10⁻⁸, less than about 0.5×10⁻⁸, less than about 9.5×10⁻⁹, less than about 9.0×10⁻⁹, less than about 8.5×10⁻⁹, less than about 8.0×10⁻⁹, less than about 7.5×10⁻⁹, less than about 7.0×10⁻⁹, less than about 6.5×10⁻⁹, less than about 6.0×10⁻⁹, less than about 5.5×10⁻⁹, less than about 5.0×10⁻⁹, less than about 4.5×10⁻⁹, less than about 4.0×10⁻⁹, less than about 3.5×10⁻⁹, less than about 3.0×10⁻⁹, less than about 2.5×10⁻⁹, less than about 2.0×10⁻⁹, less than about 1.5×10⁻⁹, less than about 1.0×10⁻⁹, less than about 0.5×10⁻⁹, less than about 9.5×10^{31 10}, less than about 9.0×10⁻¹⁰, less than about 8.5×10⁻¹⁰, or less than about 8.0×10⁻¹⁰ M. In some embodiments, the HIV-binding peptides have a K_{D} for HIV gp41 of less than 1.5×10⁻⁷, less than 1.0×¹⁰⁻⁷, less than 0.5×10⁻⁷, less than 9.5×10⁻⁸, less than 9.0×10⁻⁸, less than 8.5×10⁻⁸, less than 8.0×10⁻⁸, less than 7.5×10⁻⁸, less than 7.0×10⁻⁸, less than 6.5×10⁻⁸, less than 6.0×10⁻⁸, less than 5.5×10⁻⁸, less than 5.0×10⁻⁸, less than 4.5×10⁻⁸, less than 4.0×10⁻⁸, less than 3.5×10⁻⁸, less than 3.0×10⁻⁸, less than 2.5×10⁻⁸, less than 2.0×10⁻⁸, less than 1.5×10⁻⁸, less than 1.0×10⁻⁸, less than 0.5×10⁻⁸, less than 9.5×10⁻⁹, less than 9.0×10⁻⁹, less than 8.5×10⁻⁹, less than 8.0×10⁻⁹, less than 7.5×10⁻⁹, less than 7.0×10⁻⁹, less than 6.5×10⁻⁹, less than 6.0×10⁻⁹, less than 5.5×10⁻⁹, less than 5.0×10⁻⁹, less than 4.5×10⁻⁹, less than 4.0×10⁻⁹, less than 3.5×10⁻⁹, less than 3.0×10⁻⁹, less than 2.5×10⁻⁹, less than 2.0×10⁻⁹, less than 1.5×10⁻⁹, less than 1.0×10⁻⁹, less than 0.5×10⁻⁹, less than 9.5×10⁻¹⁰, less than 9.0×10⁻¹⁰, less than 8.5×10⁻¹⁰, or less than 8.0×10⁻¹⁰ M.

In some embodiments, the disclosed HIV-binding peptides have a K_{D} for HIV gp41 between 100 nM and 0.01 nM, between 90 nM and 0.05 nM, between 80 nM and 0.1 nM, between 70 nM and 0.5 nM, between 70 nM and 1.0 nM, or any value in between.

The disclosed HIV-binding peptides can be formulated in a pharmaceutical composition suitable for administration to a subject by any intended route of administration, as discussed in more detail below.

### IV. Pharmaceutical Compositions

Also provided herein are pharmaceutical compositions comprising a disclosed HIV-binding peptide and a pharmaceutically acceptable carrier or diluent.

The composition may be formulated for intravenous, subcutaneous, intraperitoneal, intramuscular, oral, nasal, pulmonary, ocular, vaginal, or rectal administration. In some embodiments, HIV-binding peptides are formulated for intravenous, subcutaneous, intraperitoneal, or intramuscular administration, such as in a solution, suspension, emulsion, liposome formulation, etc. More specifically, the disclosed HIV-binding peptides are formulated for intravenous or intramuscular administration. The pharmaceutical composition can be formulated to be an immediate-release composition, sustained-release composition, delayed-release composition, etc., using techniques and excipients that are known in the art.

The composition may also be formulated for oral or topical administration. For example, the disclosed HIV-binding peptides may be particularly well-suited for topical administration to the skin or mucosal surfaces (e.g., vaginal or rectal surfaces) as this route of administration may be able to provide a protective effect against initial exposure to HIV as a result of sexual contact. For instance, the disclosed HIV-binding peptides may be formulated as a gel or lubricant that could be applied directly to the vagina or rectum, or applied to a condom or incorporated into the reservoir of a condom. As discussed above, HIV-binding peptides having a recombinant secretory IgA1 or recombinant secretory IgA2 structure may be particularly useful for topical application onto mucosal surfaces.

Pharmacologically acceptable carriers for various dosage forms and routes of administration are known in the art. For example, excipients, lubricants, binders, and disintegrants for solid preparations are known; solvents, solubilizing agents, suspending agents, isotonicity agents, buffers, and soothing agents for liquid preparations are known. In some embodiments, the pharmaceutical compositions include one or more additional components, such as one or more preservatives, antioxidants, colorants, sweetening/flavoring agents, adsorbing agents, wetting agents and the like.

In some embodiments, the disclosed HIV-binding peptides may be formulated for administration by injection or infusion, such as an intravenous injection or infusion, an intramuscular injection, or a subcutaneous injection. In some embodiments, the disclosed HIV-binding peptides may be formulated for topical or oral administration.

Also disclosed herein are conjugates comprising the disclosed HIV-binding peptides, such as immunotoxin conjugates comprising a peptide and a toxin. Toxins such as the pulchellin A chain of ricin A may be covalently (i.e., chemically) conjugated to any of the disclosed HIV-binding peptides (e.g., peptide #10V or peptide #7) in order to achieve selective cytotoxicity of HIV-infected CD4+ cells. In accordance with these embodiments, HIV-infected cells that express HIV envelope protein gp41 (i.e., transmembrane gp41) can be selectively targeted for delivery of a toxin or toxic payload using conjugates of the disclosed HIV-binding peptides. Such immunoconjugates can be prepared according to methods known in the art (see, e.g., U.S. Pat. No. 9,988,438). Toxins suitable for conjugation include, but are not limited to, peptide toxins (e.g., ricin A or a fragment thereof, such as the pulchellin A chain; Pseudomonas exotoxin or a fragment thereof; pulchellin (PAC) toxins or fragments thereof; gelonin; etc.), cytotoxic drugs (e.g., epirubicin, gemcitabine, monomethyl auristatin E (MMAE), duocarmycin, maytansine, etc.), and tubulin inhibitors. The immunotoxin conjugate may comprise a linker connecting the HIV-binding peptide to the toxic agent, wherein the linker may be cleavable or non-cleavable. HIV-binding peptides or immunotoxins comprising the same may further be PEGylated in order to increase half-life or decrease immunogenicity, particularly in embodiments in which the immunotoxin comprises a peptide toxin that is potentially immunogenic. A person of ordinary skill in the art would understand how to prepare and test such an immunotoxin, as exemplified by Pincus et al., J. Virol., 91(3): e01360-16 (2017), Sadraeian et al., Sci. Reports, 7:7579, 1-12 (2017), and Pincus, J. Immunol., 170:2236-2241 (2003).

### V. Methods of Making HIV-Binding Peptides

While the disclosed HIV-binding peptides may be prepared using any known method of protein production, they also can be prepared using novel methodologies. In particular, described herein are novel methodologies for creating human neutralizing monoclonal antibodies or binding peptides, rather than “humanizing” mouse or rat antibodies/peptides. In general, this methodology allows for the development of an effective, strong, and robust library of biologics (e.g., HIV-binding peptides) that have pharmaceutical applications with significant benefits to patients or animals in the global marketplace.

In particular, using a parent hybridoma cell line, any one or more of four distinct and effective products can be produced: (1) a fully human neutralizing monoclonal antibody—directed against any pathogen (e.g., virus or bacteria)—through use in passive immunotherapy; (2) an effective humoral active vaccine that is safe and effective; (3) an oral mini-antibody peptide-based medication with an efficacy that is equivalent to the immunologic capacity of the monoclonal antibody produced by a parent hybridoma cell; and (4) an entry-fusion inhibitor that is immunologic in character and scope. The applications for these products are broad, effective and beneficial for immunotherapeutic use. For example, monoclonal antibodies for therapeutic use may be made to treat HIV (such as discussed above), as well as Rabies, Influenza A, Influenza B, Tetanus, Diphtheria, HIV-2, Anthrax, Smallpox, H1N1 Influenza, Rh (+) auto-immune disease, HTLV1 & HTLV2 Leukemia, Herpes Simplex I & II and Chronic Fatigue Syndrome, among others.

In some embodiments, the disclosed method of producing a therapeutic peptide or antibody (e.g., an HIV-binding peptide) against a targeted infectious agent (e.g., HIV) may comprises the steps of: (a) identifying an asymptomatic patient after natural infection by a target infectious agent as a donor for obtaining immune B-lymphocytes that produce high titers of plasma neutralizing antibodies directed against the target infectious agent (e.g., a virus, bacteria, fungus, or other infectious agent); (b) collecting B-lymphocytes from the patient; (c) immortalizing the human B-lymphocytes to obtain immortalized cell lines; and (d) collecting antibodies produced by the immortalized cell lines. The foregoing embodiment may optionally include the steps of (e) stabilizing and augmenting neutralizing antibody production by the immortalized cells lines; (f) screening supernatants from the immortalized cell lines for antibody production; and (g) testing the antibodies for binding against protein components of the infectious agent. The method may further comprise one or more of epitope mapping the antibodies that tested positive for binding to the infectious agent; purifying the antibodies by affinity chromatographic techniques; and in vitro testing of the antibodies to confirm neutralization reactivity against the target infectious agent at physiologic concentrations. This methodology for producing therapeutic peptides/antibodies is further exemplified in Example 2 below.

### VI. Methods of Treating HIV Infection and AIDS

As noted above, the methods of treating, preventing, and/or reducing the risk of HIV infection or AIDS described herein comprise administering to a mammalian subject in need thereof a HIV-binding peptide as disclosed herein, or a pharmaceutical composition comprising the same. In some embodiments, the methods comprise administering a HIV-binding peptide to a subject that is at risk of becoming infected with HIV, has been infected with HIV (e.g., the patient has a Stage 1 or Stage 2 HIV infection), or has developed AIDS. In some embodiments, the methods may comprise administering both a HIV-binding peptide and another compound that is useful for treating HIV/AIDS, such as one or more anti-retroviral drugs such as TDF (tenofovir), 3TC (lamivudine), FTC (emtricitabine), or EFV (efavirenz). In such embodiments, the HIV-binding peptide and the other compound(s) can be administered sequentially or simultaneously, from the same or different compositions. Thus, treatment may include administering antiretroviral drug(s) and/or other supportive treatments to address the symptoms and/or effects of HIV infection or AIDS.

In some embodiments, the method comprises administering a therapeutically effective amount of the HIV-binding peptide. As noted above, in some embodiments, a therapeutically effective amount of HIV-binding peptide is effective to reduce circulating viral load and/or to reduce, ameliorate, or eliminate one or more symptoms or effects of HIV infection or AIDS. In some embodiments, a therapeutically effective amount of HIV-binding peptide is effective to increase CD4+ cell count. The specific amount administered may depend on one or more of the age and/or weight of the subject and/or the stage or severity of the disease and/or the dosage form and route of administration.

In some embodiments, the HIV-binding peptide is administered at a dose of from about 1 to about 1000 mg/kg, about 50 mg/kg to about 850 mg/kg, about 150 mg/kg to about 750 mg/kg, about 250 mg/kg to about 650 mg/kg, or about 350 mg/kg to about 550 mg/kg. In some embodiments, the HIV-binding peptide is administered at a dose of about 1 mg/kg, about 2, mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 20 mg/kg, about 25 mg/kg, about 30 mg/kg, about 35 mg/kg, about 40 mg/kg, about 45 mg/kg, about 50 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600, about 650 mg/kg, about 700 mg/kg, about 750 mg/kg, about 800 mg/kg, about 850 mg/kg, about 900 mg/kg, about 950 mg/kg, or about 1000 mg/kg. In some embodiments, the HIV-binding peptide is administered at a dose of 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600, 650 mg/kg, 700 mg/kg, 750 mg/kg, 800 mg/kg, 850 mg/kg, 900 mg/kg, 950 mg/kg, or 1000 mg/kg. In some embodiments, the HIV-binding peptide is administered at a dose of about 3000 mg, about 3500 mg, about 4000 mg, about 4500 mg, about 5000mg, about 5500 mg, about 6000, about 6500 mg, about 7000 mg, about 7500 mg, about 8000 mg, about 8500 mg, about 9000 mg, about 9500 mg, about 10000 mg, about 10500 mg, about 11000 mg, about 11500 mg, or about 12000 mg. In some embodiments, the HIV-binding peptide is administered at a dose of 3000 mg, 3500 mg, 4000 mg, 4500 mg, 5000 mg, 5500 mg, 6000, 6500 mg, 7000 mg, 7500 mg, 8000 mg, 8500 mg, 9000 mg, 9500 mg, 10000 mg, 10500 mg, 11000 mg, 11500 mg, or 12000 mg. In some embodiments, the HIV-binding peptide is administered at a dose of up to about 10 g.

In some embodiments, the method comprises administering a single dose of HIV-binding peptide or pharmaceutical composition comprising the same, with or without another compound used for treating HIV. In other embodiments, the method comprises administering repeated doses of the HIV-binding peptide (or pharmaceutical composition) (and, optionally, other compound(s)) for a predetermined period of time, or until the symptoms or effects of HIV infection or AIDS are reduced, ameliorated, or eliminated. For instance, a subject with HIV may be evaluated for the presence and/or severity of signs and symptoms associated with HIV or AIDS, including, but not limited to, chills, fever, sweats, swollen lymph glands, weakness, weight loss, circulating viral load, CD4+ cell count, and opportunistic illnesses, and treated with an HIV-binding peptide (and, optionally, other compound(s)) until one or more of the signs/symptoms is reduced, ameliorated, or eliminated after treatment. In some embodiments, biological samples are taken from the patient to monitor viral load or CD4+ cell count at periodic intervals. In some embodiments, treatment is repeated with additional doses of the HIV-binding peptide (and, optionally, other compound(s)) if signs/symptoms/effects persist and/or if viral load remains high or CD4+ cell count remains low, and can be continued (repeated) until one or more symptoms or effects of HIV or AIDS are reduced, ameliorated, or eliminated, and/or until viral load and/or CD4+ cell count are normalized.

HIV viral load tests measure the amount of HIV in the blood. Lower levels are better than higher levels. The main goal of HIV therapies is to reduce the HIV viral load to an “undetectable” level, meaning that the HIV RNA is below the level that the test is able to count. The lower limit of HIV RNA detection depends on the test used, but in general may have a limit as low as 20-50 copies/ml. High viral loads are linked to faster disease progression. Reducing the viral load to “undetectable” levels slows or stops disease progression and prevents HIV transmission to sex partners. Treatment for HIV may suppress the virus but may not eliminate it. Even if HIV levels are not detectable, the HIV may still be in the body and could rebound to detectable levels if HIV treatment is stopped. In some embodiments, treatment with the disclosed HIV-binding peptides may be considered successful if HIV RNA copy numbers fall below 10,00 copies/ml blood on two occasions at least two weeks apart. In some embodiments, HIV is considered “controlled” or in remission if viral levels are “undetectable” by standard HIV

RNA detection methods or are as low as about 50, about 45, about 40, about 35, about 30, about 25, or about 20 copies of viral RNA/ml. Additionally or alternatively, treatment may be considered unsuccessful if HIV RNA level remain above 55,000 copies/ml or if the individual's CD4+ cell count falls below 350 cells/mm³ or 50% below the CD4+ cell count at the time that treatment commenced. Patients are considered to have progressed to AIDS when their CD4+ cell count drops below 200 cells/mm³ of blood. In contrast, normal CD4+ cell counts range from about 500 to about 1,400 cells/mm³ of blood, so CD4+ cell counts in this range are generally indicative of controlled disease.

In some embodiments, treatment may continue for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 or more days; or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more weeks; or 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more months; or 1, 2, 3, 4, 5, 6, 7, 8, 9, 10, 11, 12, 13, 14, or 15 or more years or until the subject no long experiences signs or symptoms of HIV/AIDS or is able to maintain a low viral load or high CD4+ cell count.

In some embodiments, the methods comprise administering an HIV-binding peptide (or pharmaceutical composition comprising the same) three or more times a day, twice a day, or once a day. In some embodiments, the methods comprise administering an HIV-binding peptide (or pharmaceutical composition comprising the same) once every other day, five times a week, four times a week, three times a week, twice a week, once a week, once every other week, once every three weeks, once every four weeks, once a month, once every other month, once every three months, once every four months, once every five months, once every six months, or more or less frequently. In such embodiments, the HIV-binding peptide may be a long-acting HIV-binding peptides as described above.

In some embodiments, a subject in need of treatment for HIV or AIDS is a human subject who is currently infected or at risk of becoming infected with HIV. In other words, the disclosed HIV-binding peptides may be used to treat, prevent or reduce the risk of HIV infection and/or AIDS. Without being bound by theory, it is believes that both treatment and prevention are possible through the passive immunity that the disclosed HIV-binding peptides provide to a person infected with or at risk of becoming infected with HIV. Without being bound by theory, given the ability of the disclosed HIV-binding peptides to preclude HIV from binding to and entering CD4+ cells, administration of the disclosed HIV-binding peptides may stop an HIV infection from ever taking hold in an individual into whom the virus has been introduced (i.e., the infection can be prevented). Without being bound by theory, this same mechanism would allow the disclosed HIV-binding peptides to treat active disease by preventing further replication and spread of the virus after infection.

The following examples are provided to illustrate the invention. It should be understood, however, that the invention is not to be limited to the specific conditions or details described in these examples.

### EXAMPLES

### Example 1-Treatment of a Patient With HIV

This example illustrates methods using the disclosed HIV-binding peptides in the treatment of HIV infection or AIDS.

A patient known to have or suspected of having HIV is administered a therapeutically effective amount of a pharmaceutical composition comprising an HIV-binding peptide of the present disclosure, by intravenous, intramuscular, or subcutaneous injection. The patient is evaluated for the presence and/or severity of signs and symptoms associated with HIV or AIDS, including, but not limited to, chills, fever, sweats, swollen lymph glands, weakness, weight loss, circulating viral load, CD4+ cell count, and opportunistic illnesses. Optionally, another dose of the pharmaceutical composition is administered if signs/symptoms persist and/or if viral load remains high or CD4+ cell count remains low.

### Example 2-Methods for Producing HIV-Binding Peptides

HIV-binding peptides (as well as peptides and antibodies that bind other antigens) can be produced according to the following methods.

### Step 1:

Mononuclear peripheral blood cells (including B- and T-cells) are obtained by venipuncture and Ficoll-Hypaque (F-H) technique from a patient with HIV and cultured. The supernatant from the culture of Epstein Barr Virus (EBV) Marmoset line B95-8 (day 6 of 7 day passage cycle; grown in RPMI with 10% FBS) is obtained by Millipore filtration 0.45 micron unit. The supernatant should contain approximately 10⁵ transforming units/ml.

Three (3) ml of supernatant are placed with 1×10⁷ mononuclear cells (washed ×3 with PBS after F-H separation), pelleted by centrifugation at 1200 rpm ×10 minutes. Cells are suspended with supernatant (3 ml) of the peripheral blood culture and maintained at 37° C. for 1½ hours, with intermittent resuspension. After 1½ hours incubation, 7 ml media (Iscove with 20% FBS) are added to the cells to make a concentration of roughly 1×10⁶ cells/ml.

In conducting the above steps, the following parameters are maintained: Approximately 10% of peripheral blood lymphocytes are B-cells, and 1 out of 100 B cells, approximately, are transformed by EBV.

The reacted cells-EBV supernatant are plated at 1 ml per well (24 well plate), to have 1×10⁶ cells per well. One microgram of cyclosporine A was added per well (final concentration cyclosporine A is 1 microgram per ml). Cyclosporine A is a product by Sandoz (Sandimmune).

Sandozimmune oil-like liquid solution of Cyclosporin A was extracted into the aqueous phase with ethanol 50% v/v.

### Step 2:

The second process step in the methodology provides for the fusion of EBV transformed peripheral blood immune B cells with SHMD-33 heteromyeloma for preparation of stabilized heterohybridomas.

10⁸ HIV-transformed immune B cells are mixed with 0.5 10⁸ SHMD-33—all in serum-free Iscove medium—to arrive at a ratio of 2:1. The cells are centrifuged at 1500 rpm ×10 minutes ×room temperature and the supernatant is removed.

The cell pellet is gently loosened and 1 ml of autoclaved 50% PEG (MW 1,000, in serum-free medium) is added dropwise while mixing gently. The cells should not be in contact with PEG for more than 1-2 minutes.

Nine ml of serum-free medium (warmed 37° C.) is added to the mixture, which is then centrifuged in a warm (37° C.) centrifuge at 1500 rpm ×10 minutes. The supernatant is removed and the cells are suspended gently in warm (37° C.) serum-free medium. The resuspended cells are incubated for 5 minutes at 37° C. and then centrifuged at 1500 rpm ×10 minutes ×room temperature. The cells are then resuspended in warm (37° C.) medium containing 20% FCS and incubate at 37° C. for 30 minutes. The cells are centrifuged again at 1500 rpm ×10 minutes ×room temperature, and again resuspended in Iscove medium with 20% FCS.

The cell suspension is distributed into 96-well plate at 200,000 cells/well [cell concentration 200,000 cells/100 microliters], and after 24 hours, 100 microliters 2X HAT is added with Quabain for 7 days. The cells are incubated at 37° C. without being disturbed or exposed to light for at least 3 days. After 7 days, the 100 microliters of media is removed and replaced with 100 microliters Iscove with 20% FCS (no HAT/no Quabain).

### Step 3-Cryopreservation in Liquid Nitrogen:

Subsequent to the preceding steps, the hybridoma cells are frozen using a freezing medium to keep and store at −196° C.

To prepare a cold freezing medium, 20 ml of dimethyl-sulfoxide (DMSO, Merck No. 802912, 8011 Hohebrunn, West Germany) is added to a 180 ml culture medium containing 20% serum.

Heterohybridoma cells can be centrifuging down at 5×10⁶ cells (with the mixture being kept cold), and 1 ml of cold freezing medium can be added to the resulting pellet and transferred into a sterile vial, which is stored at −70° C. for one day before being transferred into liquid nitrogen, at −196° C.

### Step 4:

In order to thaw the stored cells, remove vial out of liquid nitrogen and thaw quickly in a 37° C. water bath. As soon as the solution is liquefied, remove cells with a Pasteur pipette and transfer them into a 50 mil tube containing cold growth medium.

Pellet the cells and wash again with 50 ml cold medium. The cells can be grown at different dilutions: 2.5×10⁵, 5×10⁴ viable cells per ml (as judged by trypan blue). This should be done because the trypan blue count often does not reflect a possible large fraction of cells which might die after a one-day culture period.

Using the methodology of the present Example, fully human (or fully animal) monoclonal antibodies are produced for use in treating patients with (1) virus, such as HIV, (2) bacteria, (3) fungus or (4) other infectious agents.

While preferred materials and components have been described, the method is not limited specifically by these materials but includes the substitution of equivalent materials.

### Example 3-Determination of Binding Affinity of HIV-Binding Peptides #9-10

Various ELISA-based assays were carried out in order to determine the binding affinity of HIV-binding peptides to HIV, and to the epitope having the amino acid sequence of SEQ ID NO:1 in particular.

In a first study, an ELISA was prepared in which the linear (reduced) form of the 15-mer peptide LGIWGCSGKLICTTT (SEQ ID NO:1) was coated onto a solid support and contacted with various concentrations of peptides #1, #2, #3, #4, #5, #6, #7, #8, #9Q, #10Q, and #11Q. A mouse, anti-human IgG Fc-HRP at 0.2 μg/ml was used as a secondary antibody. The following table shows the results of the study.

TABLE 5
ELISA Binding Data

| Antibody | 1000 ng/ml | 333 ng/ml | 111 ng/ml | 37 ng/ml | 12 ng/ml | Neg. Cont. | Coating Antigen | Secondary antibody |
|---|---|---|---|---|---|---|---|---|
| #1 | 0.079 | 0.096 | 0.076 | 0.098 | 0.08 | 0.096 | U1731DB060-1, | Mouse anti-human |
| #2 | 0.068 | 0.076 | 0.188 | 0.074 | 0.065 | 0.075 | 15-mer peptide | IgG Fc-HRP, |
| #3 | 0.072 | 0.077 | 0.072 | 0.074 | 0.087 | 0.083 | LGIWGCSGKLICTTT | 0.2 μg/ml |
| #4 | 0.07 | 0.079 | 0.073 | 0.077 | 0.073 | 0.091 | 1 ug/ml in DTT | |
| #5 | 0.081 | 0.086 | 0.071 | 0.081 | 0.082 | 0.078 | | |
| #6 | 0.079 | 0.078 | 0.08 | 0.072 | 0.077 | 0.087 | | |
| #7 | 0.09 | 0.092 | 0.084 | 0.098 | 0.096 | 0.088 | | |
| #8 | 0.076 | 0.073 | 0.1 | 0.089 | 0.094 | 0.088 | | |
| #9 | 0.745 | 0.322 | 0.171 | 0.122 | 0.111 | 0.105 | | |
| #10 | 1.714 | 0.863 | 0.377 | 0.209 | 0.14 | 0.084 | | |
| #11 | 0.441 | 0.241 | 0.127 | 0.119 | 0.092 | 0.104 | | |

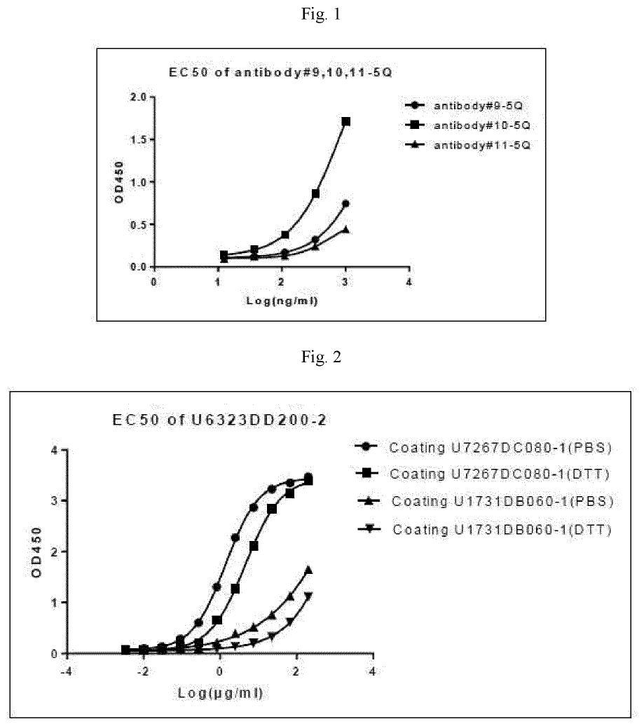

This data is shown in graphical form in FIG. 1 for #9Q, #10Q, and #11Q, which exhibited the greatest binding affinity.

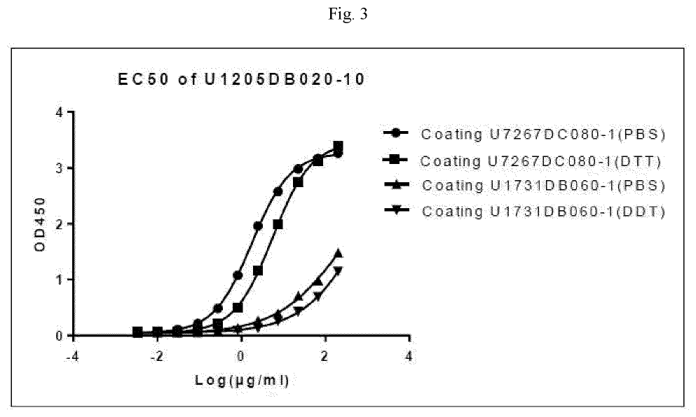

Variants #9V, #10V, and #11V were prepared with substitutions in the heavy chain. These variants comprised amino acids sequences nearly identical to #9Q, #10Q, and #11Q, respectively, with the only distinction being a substitution of glutamine (Q) for valine (V) at the 5 position of the heavy chain. Exemplary peptides #10Q and #10V were tested in indirect ELISA assays to determine the EC50 values for binding to the epitope having the amino acid sequence of SEQ ID NO:1 and HIV gp41. The results of these studies are shown in Tables 6 and 7 below, as well as FIGS. 2 and 3. Target proteins U7267DC080-1 and U173DB060-1 correspond to recombinant gp41 and the 15-mer peptide LGIWGCSGKLICTTT (SEQ ID NO:1), respectively.

TABLE 6
ELISA Binding Data for #10V

| | | |
|---|---|---|
| Concentration of | Coating | Coating |
| U6323DD200-2 | U7267DC080-1 | U173DB060-1, |
| antibody#10-5V | Target protein | Target peptide |
| μg/ml | 2 μg/ml | 2 μg/ml |

| | | | | |
|---|---|---|---|---|
| 200.000 | 3.469 | 3.394 | 1.649 | 1.115 |
| 66.667 | 3.351 | 3.140 | 1.129 | 0.618 |
| 22.222 | 3.233 | 2.845 | 0.756 | 0.329 |
| 7.407 | 2.870 | 2.116 | 0.514 | 0.202 |
| 2.469 | 2.276 | 1.270 | 0.396 | 0.141 |
| 0.823 | 1.308 | 0.660 | 0.212 | 0.094 |
| 0.274 | 0.606 | 0.211 | 0.105 | 0.068 |
| 0.091 | 0.285 | 0.130 | 0.089 | 0.061 |
| 0.030 | 0.136 | 0.087 | 0.064 | 0.058 |
| 0.010 | 0.095 | 0.067 | 0.093 | 0.059 |
| 0.003 | 0.080 | 0.065 | 0.062 | 0.058 |
| 0.000 | 0.076 | 0.077 | 0.062 | 0.060 |
| EC50(μg/ml) | 1.394 | 4.592 | / | / |
| Coating buffer | PBS | DTT | PBS | DTT |

TABLE 7
ELISA Binding Data for #10Q

| | | |
|---|---|---|
| Concentration of | Coating | Coating |
| U1205DB020-10, | U7267DC080-1, | U173DB060-1 |
| antibody#10-5Q | Target protein | Target peptide |
| μg/ml | 2 μg/ml | 2 μg/ml |

| | | | | |
|---|---|---|---|---|
| 200.000 | 3.265 | 3.396 | 1.482 | 1.152 |
| 66.667 | 3.173 | 3.125 | 0.984 | 0.695 |
| 22.222 | 2.983 | 2.737 | 0.713 | 0.429 |
| 7.407 | 2.577 | 1.998 | 0.395 | 0.263 |
| 2.469 | 1.962 | 1.162 | 0.269 | 0.135 |
| 0.823 | 1.080 | 0.498 | 0.139 | 0.092 |
| 0.274 | 0.488 | 0.212 | 0.084 | 0.074 |
| 0.091 | 0.216 | 0.110 | 0.063 | 0.060 |
| 0.030 | 0.112 | 0.075 | 0.056 | 0.059 |
| 0.010 | 0.077 | 0.065 | 0.055 | 0.055 |
| 0.003 | 0.068 | 0.067 | 0.053 | 0.053 |
| 0.000 | 0.065 | 0.092 | 0.060 | 0.057 |
| EC50(μg/ml) | 1.779 | 5.541 | / | / |
| Coating buffer | PBS | DTT | PBS | DTT |

These studies indicated that both #10V and #10Q bound to HIV gp41 more strongly than to the epitope having the amino acid sequence of SEQ ID NO:1, and that #10V bound to both the epitope having the amino acid sequence of SEQ ID NO:1 and HIV gp41 with better affinity than #10Q. Specifically, the EC50 value for #10V against gp41 was 1.394 μg/ml, while it was 1.779 μg/ml for #10Q. Similarly, the EC50 value for #10V against SEQ ID NO:1 was 4.592 μg/ml, while it was 5.541 μg/ml for #10Q. (Lower EC50 values represent stronger binding.)

### Example 4-Determination of Binding Affinity of HIV-Binding Peptides #1-8

HIV-binding peptides #1-8 were tested in ELISA assays to determine their relative affinity rankings to the HIV antigen gp41. HIV-binding peptides #1-8 are all recombinant, fully human immunoglobulin proteins with varied light chain peptide sequences paired with identical heavy chain peptide sequences. For the purposes of the ELISA, a solid support was coated with recombinant antigen gp41 (i.e., U1916DG190-1) at concentrations of 2 μg/ml (100 μl/well). PBS at pH 7.4 was used as a coating buffer and mouse anti-human IgG Fc-HRP was used as a secondary antibody.

TABLE 8
Affinity Binding Rankings of HIV-Binding Peptides #1-8

| Affinity | | |
|---|---|---|
| ranking | antibody | EC50(μg/ml) |
| 1 | U1205DB020-7 | 106.7 |
| 2 | U1205DB020-6 | 222.4 |
| 3 | U1205DB020-5 | 249.7 |
| 4 | U1205DB020-2 | 283.4 |
| 5 | U1205DB020-1 | 392.1 |
| 6 | U1205DB020-4 | 1505 |
| 7 | U1205DB020-8 | 2705 |
| 8 | U1205DB020-3 | / |

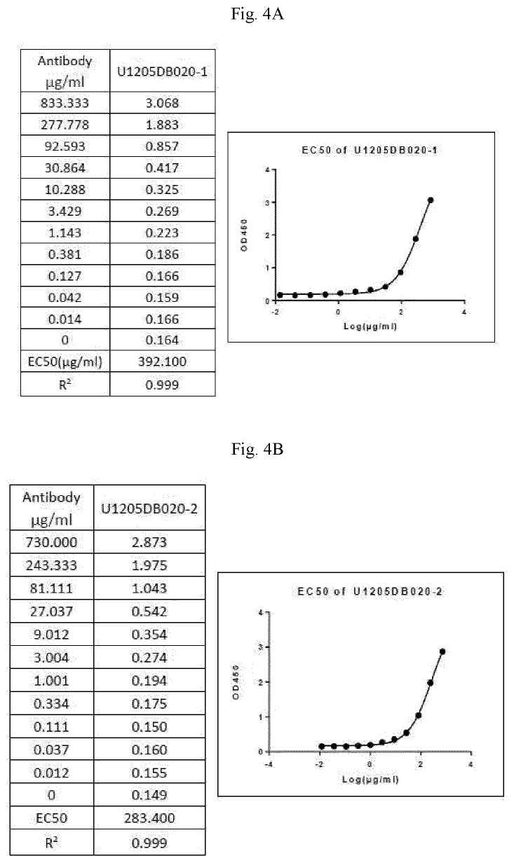

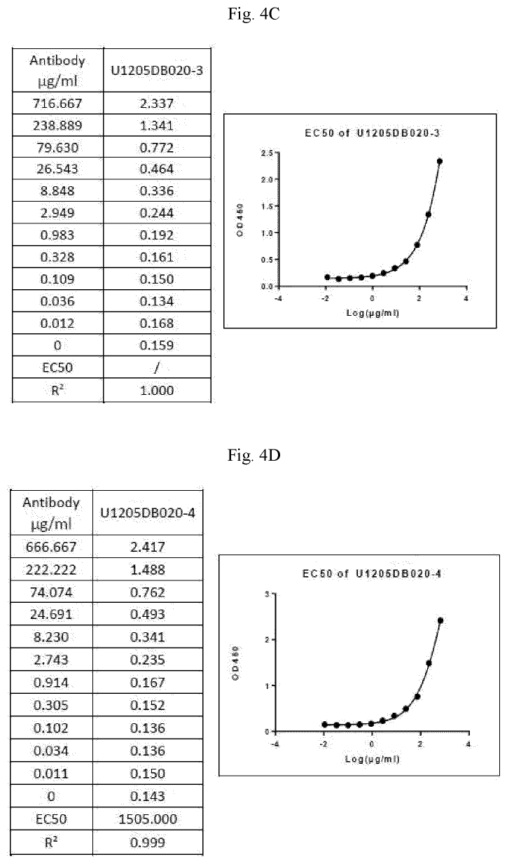

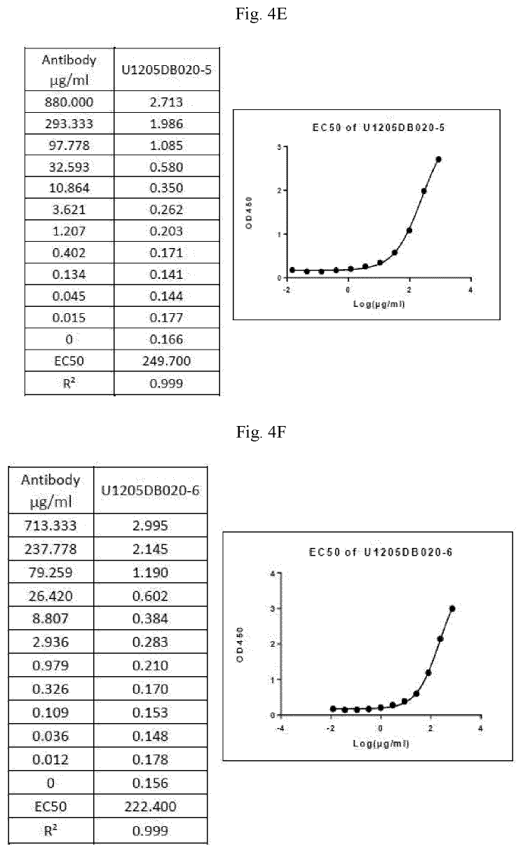

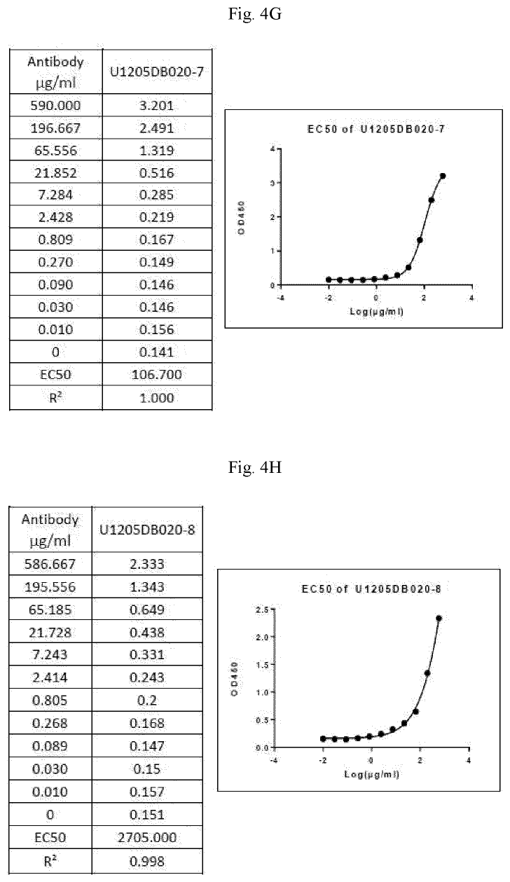

The results showed that HIV-binding peptide #7 [U1205DB020-7] possesses the best EC₅₀ (at 106.700 μg/ml) compared to HIV-binding peptides #1-8 when directed against recombinant gp41 in PBS. The results for each of the individual peptides are shown in FIGS. 4A-4H.

### Example 5-Neutralization and Binding Studies

Three purified binding peptides, #7 (i.e., two light chains comprising SEQ ID NO: 24 and two heavy chains comprising SEQ ID NO: 25), #10Q (i.e., a nanobody heavy chain dimer comprising SEQ ID NO: 10), and #10V (i.e., a nanobody heavy chain dimer comprising SEQ ID

NO: 11)—were tested for efficacy in neutralization against HIV isolates SF-162 clade B clinical isolate in vitro, using PBMC based neutralization. The results are shown in Table 9, below.

TABLE 9
EC50 and Binding Data for #7, #10Q, and #10V

| | GenScript Binding | | IC value vs SF 162 |
|---|---|---|---|
| | EC50 vs rgp41 | Pincus FACS | (μg/ml) in PBMC |
| Peptide | (μg/mL) | Binding* | based in vitro assays |
| #7 | 106.7 μg/ml | 25,941/25,039 | IC70 = 0.50 μg/ml |
| #10Q | 1.779 μg/ml | 120,324/110,043 | IC90 = 0.05 μg/ml |
| #10V | 1.394 μg/ml | 56,625/29,906 | IC70 = 0.50 μg/ml |

*Fluorescent (FACS) counts in both the absence (x) or the presence (y) of sCD4 (x/y)

For the FACS binding studies, the CD4+ cells that were infected with HIV expressed HIV viral envelope proteins on the cell membrane surfaces, e.g., gp160=gp41 and gp 120. Binding of proteins #7, #10Q, and #10V was tested in both presence and absence of soluble CD4 (sCD4). The positive FACS binding study shows that all three proteins bind to gp41 (the HIV viral envelope protein which contains SEQ ID NO: 2) on CD4+ cell membrane.

Interestingly, greater fluorescence binding to CD4 cell-surface gp41 was demonstrated in the absence of sCD4. Typically, most anti-gp41 antibodies have shown a marked increase in fluorescence in the presence of sCD4. Thus, the fact that the three disclosed proteins exhibited greater fluorescence binding to CD4 cell-surface gp41 in the absence of sCD4 was unexpected.

### Example 6-Testing Class Switched Antibodies

For the purposes of this example, recombinant IgA1 and IgA2 class switched antibodies were created and tested. The class switched antibodies created for this example were nanobody heavy chain dimers, with the IgA1 comprising two heavy chains of SEQ ID NO: 35 and the IgA2 comprising two heavy chains of SEQ ID NO: 36.

Recombinant dimeric secretory IgA1 and IgA2, each comprising two variable heavy domains of SEQ ID NO: 34, were prepared and tested using an ELISA binding studies directed against recombinant gp41 (in PBS). The EC50 values were: 1183 ng/ml (1.183 μg/ml) for the IgA1 antibody and 1293 ng/ml (1.293 μg/ml) for the IgA2 antibody.

These data indicate that IgA class switched antibodies may be useful a therapy for providing protective immunological defense against initial exposure or infection with HIV.

What is claimed is:

1. A recombinant peptide that binds to human immunodeficiency virus (HIV), comprising a heavy chain variable region comprising at least three complementarity determining regions (CDRs) including:
(a) CDR1 comprising the amino acid sequence of GFTFSSY (SEQ ID NO:3);
(b) CDR2 comprising the amino acid sequence of SYDGSN (SEQ ID NO:4); and
(c) CDR3 comprising the amino acid sequence of DRFSAVASTPTYHNYFYMDV (SEQ ID NO:5);
wherein the peptide binds to an epitope of HIV comprising the amino acid sequence KLIC (SEQ ID NO: 2) and prevents binding between HIV and CD4+ cells.

2. The recombinant peptide of claim 1, wherein the heavy chain variable region comprises SEQ ID NO:33 or SEQ ID NO:34.

3. The recombinant peptide of claim 1 or 2, wherein the peptide is a recombinant immunoglobulin or fragment thereof.

4. The recombinant peptide of claim 3, wherein the recombinant immunoglobulin is an immunoglobulin heavy chain dimer.

5. The recombinant peptide of claim 3, wherein the recombinant immunoglobulin is an immunoglobulin heavy chain monomer.

6. The recombinant peptide of claim 3, wherein the peptide is a single-domain antibody.

7. The recombinant peptide of any one of claims 1-6, wherein the peptide comprises SEQ ID NO:6 or SEQ ID NO:7.

8. The recombinant peptide of any one of claims 1-6, wherein the peptide comprises SEQ ID NO:8 or SEQ ID NO:9.

9. The recombinant peptide any one of claims 1-6, wherein the peptide comprises SEQ ID NO:10 or SEQ ID NO:11.

10. The recombinant peptide of any one of claims 1-9, wherein the peptide comprises a CH2 domain and a CH3 domain.

11. The recombinant peptide of claim 10, wherein the CH2 domain comprises SEQ ID NO:29 or SEQ ID NO:32; and the CH3 domain comprises SEQ ID NO:30.

12. The recombinant peptide of claim 10 or 11, wherein the peptide does not comprise a CH1 domain.

13. The recombinant peptide of claim 10 or 11, wherein the peptide comprises a CH1 domain.

14. The recombinant peptide of claim 13, wherein the CHI domain comprises SEQ ID NO:28 or SEQ ID NO:31.

15. The recombinant peptide of any one of claims 1-14, wherein the peptide comprises SEQ ID NO:33.

16. The recombinant peptide of any one of claims 1-5, wherein the peptide comprises at least one constant domain from a human IgA.

17. The recombinant peptide of claim 16, wherein the IgA is a secreted IgA.

18. The recombinant peptide of claim 16 or 17, wherein the IgA is an IgA1 or an IgA2.

19. The recombinant peptide of any one of claims 1-18, wherein the epitope of HIV comprises the amino acid sequence LGIWGCSGKLICTTT (SEQ ID NO:1) or a fragment thereof comprising the amino acid sequence KLIC (SEQ ID NO:2).

20. The recombinant peptide of any one of claims 1-19, wherein the peptide exhibits an optical density of at least about 0.441, at least about 0.745, or at least about 1.714 in an ELISA binding assay at 1000 ng/ml of the recombinant peptide.

21. The recombinant peptide of any one of claims 1-20, wherein the peptide is conjugated to a peptide toxin, a cytotoxic drug, or a tubulin inhibitor.

22. The recombinant peptide of claim 21, wherein the peptide toxin is selected from ricin A or a fragment thereof, a Pseudomonas exotoxin or a fragment thereof; a pulchellin toxins or a fragment thereof; or gelonin.

23. The recombinant peptide of claim 21, wherein the cytotoxic drug is selected from epirubicin, gemcitabine, monomethyl auristatin E (MMAE), duocarmycin, or maytansine.

24. The recombinant peptide of any one of claims 1-23, wherein the peptide is PEGylated.

25. A method of treating, preventing, or reducing the risk of HIV infection in a subject in need thereof, comprising administering to the subject a therapeutically effective amount of a recombinant peptide according to any one of claims 1-24.

26. The method of claim 25, wherein administration of the recombinant peptide provides the subject with passive immunity to HIV.

27. The method of claim 25 or 26, wherein the recombinant peptide is administered orally or parenterally.

28. The method of claim 25 or 26, wherein the recombinant peptide is administered topically to a mucosal membrane or skin.

29. The method of claim 28, wherein administration of the recombinant peptide provides the subject with pre-exposure prophylaxis.

30. A recombinant peptide according to any one of claims 1-24 for treating, preventing, or reducing the risk of HIV infection in a subject in need thereof.

31. Use of a recombinant peptide according to any one of claims 1-24 in the preparation of a medicament for treating, preventing, or reducing the risk of HIV infection in a subject in need thereof.

32. A method of preparing a recombinant peptide that binds to HIV, comprising:
(a) identifying an asymptomatic patient that has been infected with HIV as a donor for obtaining immune B-lymphocytes that produce high titers of HIV-neutralizing antibodies;
(b) collecting the B-lymphocytes from the patient;
(c) immortalizing the B-lymphocytes; and
(d) collecting antibodies produced by the immortalized cells.

33. The method of claim 32, further comprising screening supernatants from the immortalized B-lymphocytes for HIV-binding antibodies.

34. The method of claim 32 or 33, further comprising testing the antibodies for binding HIV.

35. The method of claim 34, further comprising epitope mapping the antibodies that tested positive for binding to HIV.

36. The method of any one of claims 32-35, wherein purifying the antibodies from the cell culture supernatant comprises affinity chromatographic techniques.

37. The method of any one of claims 32-36, further comprising testing the antibodies in vitro to confirm neutralization reactivity at physiologic concentrations against HIV.

38. The method of any one of claims 32-37, wherein immortalizing the B-lymphocyte comprises fusing a B-lymphocyte with a heteromyeloma cell in order to produce a heterohybridoma cell.

39. A recombinant peptide that binds to human immunodeficiency virus (HIV), comprising at least three complementarity determining regions (CDRs) including:
(a) CDR1 comprising the amino acid sequence of GFTFSSY (SEQ ID NO:3);
(b) CDR2 comprising the amino acid sequence of SYDGSN (SEQ ID NO:4); and
(c) CDR3 comprising the amino acid sequence of DRFSAVASTPTYHNYFYMDV (SEQ ID NO:5);
wherein the recombinant peptide is produced according to the method of any one of claims 32-38.
